(19) United States
(12) Patent Application Publication
(10) Pub. No.: US 2024/0325652 A1
(43) Pub. Date: Oct. 3, 2024

(54) Components and subassemblies for medicament delivery devices

(71) Applicant: SHL Medical AG, Zug (CH)

(72) Inventors: Christopher Foster, Zug (CH); Ming-Ting Yin, Taoyuan City (TW)

(21) Appl. No.: 18/709,855

(22) PCT Filed: Nov. 9, 2022
(86) PCT No.: PCT/EP2022/081190

§ 371 Date: May 14, 2024

(30) Foreign Application Priority Data
Nov. 23, 2021 (EP) 21210078.8

(51) Int. Cl. A61M 5/315; A61M 5/20

(52) U.S. Cl. CPC A61M 5/31501; A61M 2005/202; A61M 2005/2026

(57) ABSTRACT

The present disclosure provides an inner tubular body for a medicament delivery device. The inner tubular body extends along a longitudinal axis from a proximal end to a distal end. The inner tubular body includes a distal part and a proximal part. The distal part includes a flexible arm extending distally, the flexible arm comprises a first spring support at the distal end of the flexible arm and a plunger rod support at the distal end of the flexible arm. The first spring support is inclined in a radially inward direction and is configured to support a distal end of a drive spring of the medicament delivery device when the medicament delivery device is in a non-activated state. The distal part further includes a second spring support extending distally and being configured to support the distal end of the drive spring of the medicament delivery device when the medicament delivery device is in an activated state. The plunger rod support is configured to engage with a plunger rod of the medicament delivery device in order to inhibit a proximal movement of the plunger rod when the medicament delivery device is in the non-activated state. The first spring support is configured to interact with an activation button that is at a distal end of the medicament delivery device. Triggering of the activation button allows a radial outward deflection of the flexible arm thereby disengaging the plunger rod support from the plunger rod and disengaging the first spring support from the distal end of the drive spring.

### CROSS-REFERENCE TO RELATED APPLICATIONS

The present application is a U.S. National Phase Application pursuant to 35 U.S.C. § 371 of International Application No. PCT/EP2022/081190 filed Nov. 9, 2022, which claims priority to European Patent Application No. 21210078.8 filed Nov. 23, 2021. The entire disclosure contents of these applications are herewith incorporated by reference into the present application.

### TECHNICAL FIELD

The present invention relates to medicament delivery devices. In particular, the present invention relates to components and subassemblies for medicament delivery devices.

### BACKGROUND

A number of medical conditions require reliable provision of a medicament from a medicament delivery device. The medicament is typically comprised in a medicament container within the medicament delivery device, the medicament container being configured to expel the medicament via some type of delivery member, such as a medicament delivery member or a nozzle.

Medicament delivery devices, especially disposable single-dose auto-injectors, are often intended to be used by users in a home environment, enabling the user to self-apply a medical substance, for example insulin or hormones, without a medically trained person being present. As a result, easy and safe handling of such devices is a key design aspect. Thus, depending on the intended use and type of medicament, medicament delivery devices having a varying degree of automatic functions have been developed to facilitate injection of medicaments in a reliable and safe manner.

These days, a number of different medicament delivery devices exist, including various types of injectors (for example pen injectors, autoinjectors and on-body devices). Although many of these devices have enabled major improvements in the management of a number of medical conditions, various drawbacks do still exist in the current technology.

Auto-injectors with a penetration function usually comprise a housing, a container holder carrying a medicament container with a delivery member and a plunger rod as well as a compression spring providing a force to move the plunger rod with respect to the housing and/or container holder. Further, the medicament delivery device often comprises some type of delivery member cover configured to cover the delivery member prior to a medicament delivery operation, as well as subsequent to the medicament delivery operation. For the latter, the medicament container can be held in a retracted position within the medicament delivery device, while the delivery member, e.g. a medicament delivery member, is prevented from being exposed in order avoid accidental re-use of the device.

Moreover, existing auto-injectors usually comprise an activation button. Prior to actuation, the auto-injector is in a secured state, and the user needs to press the activation button (for example an activation button) which initiates the actuation. When said auto-injectors are actuated, the container holder and the plunger rod, which are slidable in the housing, are jointly driven towards the user's skin by the force of the compression spring, which causes the medicament delivery member to penetrate the user's skin. Usually the activation button has some play to move after the activation and such play may give rise to a rattling sound.

It has therefore been appreciated that improvements can be made to certain components and subassemblies for medicament delivery devices.

### BRIEF DESCRIPTION

The present disclosure is applicable to a number of medical devices, including, but not limited to, devices that automatically, semi-automatically or manually deliver one or more doses of medicament through injection (needle and needleless), inhalation, infusion, atomization, drops, patches, and implants. Incorporating one or more automatic feedback mechanisms into these medical devices ensures that the user of the device will be notified of the beginning of the medicament delivery sequence.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis. Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

The term automatic in conjunction with medical injection device means that the injector device is able to perform the injection without the user of the device delivering the force needed to expel the medicament during delivery. The force is delivered automatically by a resilient force such as a spring or an electric motor or any other suitable means for providing the delivery force.

Further, in the following description, the wording medicament delivery device will be used. In this context, medicament delivery devices may include a number of devices capable of delivering certain doses of medicament to a user. The medicament delivery devices may be either disposable or re-usable and may be provided with medicament containers suitably arranged for delivering specific drugs in specific forms.

Although the medical device described below is a medicament delivery device configured as a disposable single-use, pen-type injector, such as an auto-injector, any type of automatic medicament delivery device could incorporate the actuation mechanisms of the present disclosure, including, but not limited to, inhalers or eye dispensers. Likewise, the medical device may be a training device that replicates a medicament delivery device.

A first aspect of the invention concerns an inner tubular body for a medicament delivery device, wherein the inner tubular body extends along a longitudinal axis L from a proximal end to a distal end. The inner tubular body comprising a distal part and a proximal part, wherein the distal part comprises a flexible arm extending distally, wherein the flexible arm comprises a first spring support at the distal end of the flexible arm and a plunger rod support at the distal end of the flexible arm. The first spring support is inclined in a radially inward direction and is configured to support a distal end of a drive spring of the medicament delivery device when the medicament delivery device is in a non-activated state S1. The distal part further comprises a second spring support extending distally and being configured to support the distal end of the drive spring of the medicament delivery device when the medicament delivery device is in an activated state S2. The plunger rod support is configured to engage with a plunger rod of the medicament delivery device in order to inhibit a proximal movement of the plunger rod when the medicament delivery device is in the non-activated state, and wherein the first spring support is configured to interact with an activation button that is at a distal end of the medicament delivery device, with triggering of said activation button allowing a radial outward deflection of the flexible arm thereby disengaging the plunger rod support from the plunger rod and disengaging the first spring support from the distal end of the drive spring.

An advantage of supporting the drive spring when the device is in the activated state is the prevention of a rattling movement of the drive spring.

Optionally, the flexible arm and the second spring supports are spaced apart so that the flexible arm can move relative to the second spring supports.

Optionally, the second spring support comprises a second spring support surface, spaced apart in the distal direction D2 with respect to a first spring support surface of the first spring support such that, when in the non-activated state S1, the distal end of the drive spring is supported by the first spring support surface and the distal end of the drive spring is spaced apart by a distance d3 from the second spring support surface.

Optionally, when the device is in the activated state S2 the flexible arm is configured to flex outwardly and disengage the drive spring in the distal direction D2 such that the distal end of the drive spring abuts the second spring support surface thereby the distal end of the drive spring has been displaced by the distance d3 in the distal direction D2.

An advantage of spacing apart the flexible arms from the second spring support is that it allows for kinematic decoupling of the flexible arms from the second spring support, and spacing apart the first and second spring support from each other by a distance d3 prevents buckling and rattling of the drive spring, i.e. further movement of the drive spring is inhibited after expansion of the drive spring.

Optionally each plunger rod support has a radially inwards extending protrusion configured to form a stop surface for engagement with a distal portion of the plunger rod.

Optionally, each first spring support comprises a protrusion or step extending radially inwards and having a proximally facing support surface configured to support the distal end of the drive spring.

Optionally, when in the activated state S2 the distal end of the drive spring exerts a radially outwardly directed force on the flexible arm such that the flexible arm is further pushed outwardly, thereby abutting inwardly inclined surfaces of inwardly extending protrusions of an activation button such that the activation button is prevented from moving in the distal direction D2.

Optionally, the flexible arm and the at least two second spring supports are alternately arranged in the circumferential direction.

A second aspect concerns a rear subassembly for a medicament delivery device, the rear subassembly comprising a distal housing, an inner tubular body according to any one of the preceding options, wherein the tubular body is coaxially arranged within the distal housing and distally movable in relation to the distal housing; a plunger rod coaxially arranged within the inner tubular body and releasably connected to the plunger rod support of the inner tubular body; a drive spring coaxially arranged between the plunger rod and second spring supports of the inner tubular body, wherein a distal end of the drive spring is supported by the first spring support when in a first non-activated state S1 or the distal end of the drive spring is supported by the second spring supports when in a second activated state S2 and a proximal end of the drive spring is supported by a proximal ledge of the plunger rod; an activation button connected to the distal housing, wherein the activation button comprises a radially inwardly extending protrusion, the radially inwardly extending protrusion comprising a radially inwardly inclined surface, wherein the inclined surface is facing in the distal direction D2, the protrusion comprising a step extending radially inwards, the step having a proximally facing support surface configured to support a radially outwardly extending protrusion of the flexible arm when the medicament delivery device is in the non-activated state S1; a distal spring arranged between the activation button and the inner tubular body, wherein the distal spring is configured to apply a biasing force on the inner tubular body.

Optionally, the activation button comprises a guide hole along the longitudinal axis of the medicament delivery device configured to axially guide the distal end of the plunger rod when the inner tubular body is distally displaced.

Optionally, the activation button comprises a displacement structure configured to interact with an inclined distal end surface of the flexible arm in order to radially deflect the flexible arm such that the plunger rod support is disengaged from the plunger rod when the inner tubular body is distally displaced.

Optionally, the displacement structure is rigid and is radially inclined, preferably with the same angle of inclination as the inclined end surface of the flexible arm.

A third aspect concerns a medicament delivery device comprising a distal subassembly and a proximal subassembly, wherein the distal subassembly is a rear subassembly according to one of above options of the second aspect.

Optionally, the proximal subassembly comprises a proximal housing connectable to the distal housing; a rotator coaxially connected to a distal inner part of the proximal housing and movable in relation to the proximal housing; a displaceable medicament delivery member cover partially arranged within the proximal housing; a medicament container holder connected to the medicament delivery member cover and configured to receive a medicament container; a medicament delivery member cover spring disposed between the medicament delivery member cover and the medicament container holder and configured to apply a biasing force to the medicament delivery member cover, wherein the medicament delivery member cover is axially movable inside at least the proximal housing such that a distal end of the medicament delivery member cover contacts the contact surface of the inner tubular body whereby the inner tubular body is distally displaced.

Optionally, the medicament delivery device is a disposable single-dose auto-injector.

These and other aspects of and advantages with the present invention will become apparent from the following detailed description and from the accompanying drawings.

### BRIEF DESCRIPTION OF THE DRAWINGS

Embodiments of the Invention Will Now be Described by Way of Example Only and with Reference to the Accompanying Drawings, in which:

FIG. 1 shows a perspective view of a medicament delivery device 10.

FIG. 2A shows a perspective view of the cross-sectional planes P1 and P2 perpendicular to each other and with respect of the longitudinal axis L.

FIG. 2B shows a rear subassembly 20 of the medicament delivery device 10.

FIG. 3A shows a detailed perspective view of the rear subassembly 20 in an initial, non-activated state without the activation button.

FIG. 3B shows a perspective view of the rear subassembly 20 in an initial, non-activated state without the activation button.

FIG. 3C shows a detailed perspective view of the rear subassembly 20 in an intermediate activated state without the activation button.

FIG. 3D shows a detailed perspective view of the rear subassembly 20 in a final, activated state without the activation button.

FIG. 3E shows a perspective view of the rear subassembly 20 in a final, activated state without the activation button.

FIG. 4A shows a cross-sectional view along the plane P1 of the rear subassembly 20.

FIG. 4B shows a cross-sectional view along the plane P2 of the rear subassembly 20.

FIG. 5 shows a cross-sectional view along the plane P1 of the rear subassembly 20 in an initial or default state.

FIG. 6 shows a cross-sectional view along the plane P1 of the rear subassembly 20 in a final or activated state.

FIG. 7 shows a cross-sectional view along the plane P2 of the rear subassembly 20 in an initial or default state.

FIG. 8 shows a cross-sectional view along the plane P2 of the rear subassembly 20 in a final or activated state.

### DETAILED DESCRIPTION

Various modifications to the embodiments described are possible and will occur to those skilled in the art without departing from the invention which is defined by the following claims.

These and other aspects of and advantages with the present invention will become apparent from the following detailed description and from the accompanying drawings.

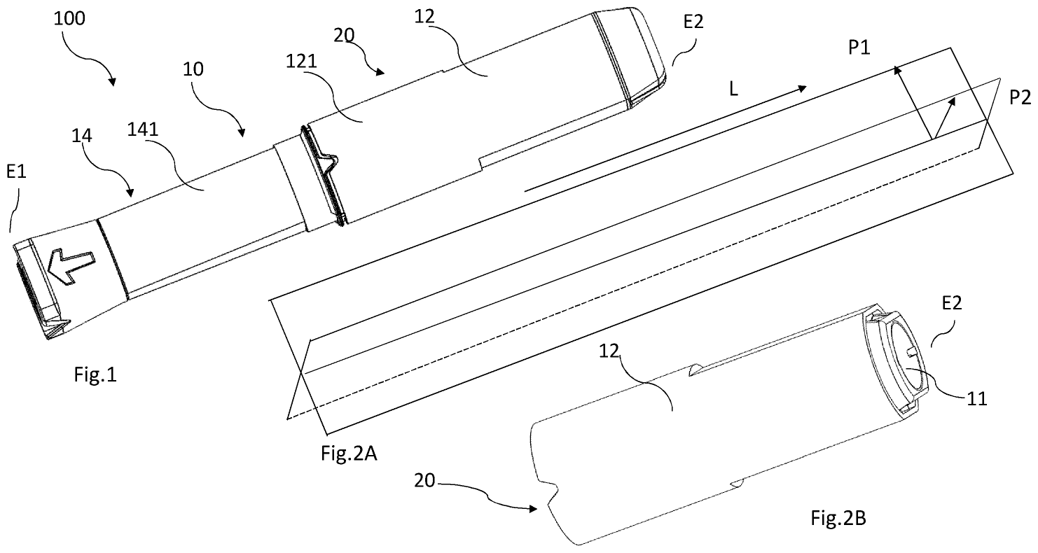

FIG. 1 schematically illustrates a medicament delivery device 10 according to one embodiment of the present disclosure, wherein the device 10 is fully assembled. Hereinafter, the terms “medicament delivery device” and “device” will be used interchangeably.

The device 10 can be considered as two sub-assemblies, a rear subassembly 20, namely a so-called power pack and a so-called front assembly 14. The rear subassembly 20, i.e. the power pack, and the front assembly 14 are arranged such that they both extend along a longitudinal axis L, from a proximal end E1 to a distal end E2. The rear subassembly 20 comprises a tubular outer body 12 and an activation button 11, both extending along the longitudinal axis L. The activation button 11 is at least partially arranged within the tubular outer body 12 and is movable along the longitudinal axis L. Here, “movable” implies that the activation button can, if not blocked by any other component of the rear subassembly 20, be pushed at least in a proximal direction D1 relative to the tubular outer body 12. Such pushing of the activation button 11 can be achieved e.g. by a user using a finger, such as a thumb, to apply a force on the activation button in the proximal direction.

When arranged as in FIG. 1, the longitudinal axis of each sub-assembly aligns with the longitudinal axis L of the device as a whole. Likewise, the distal end of the rear subassembly 20 is also the distal end E2 of the device 10, while the proximal end of the rear subassembly 20 is the proximal end E1 of the device 10. The proximal end of the rear subassembly 20 can be understood as the end of the rear subassembly 20 being closest to the front assembly 14 and, similarly, the distal end of the front assembly 14 can be understood as the end of the front assembly 14 being closest to the activation button 11.

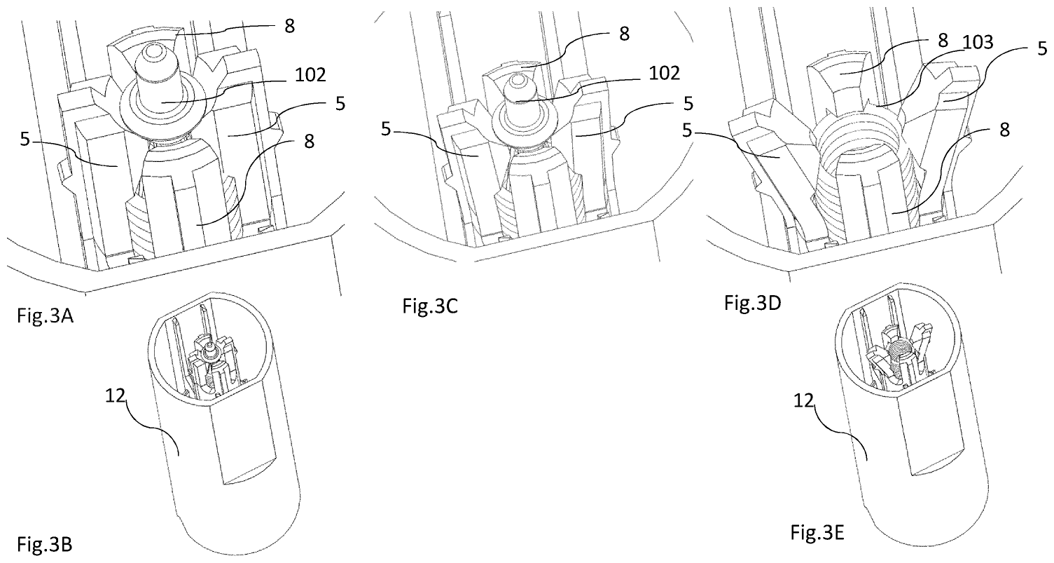

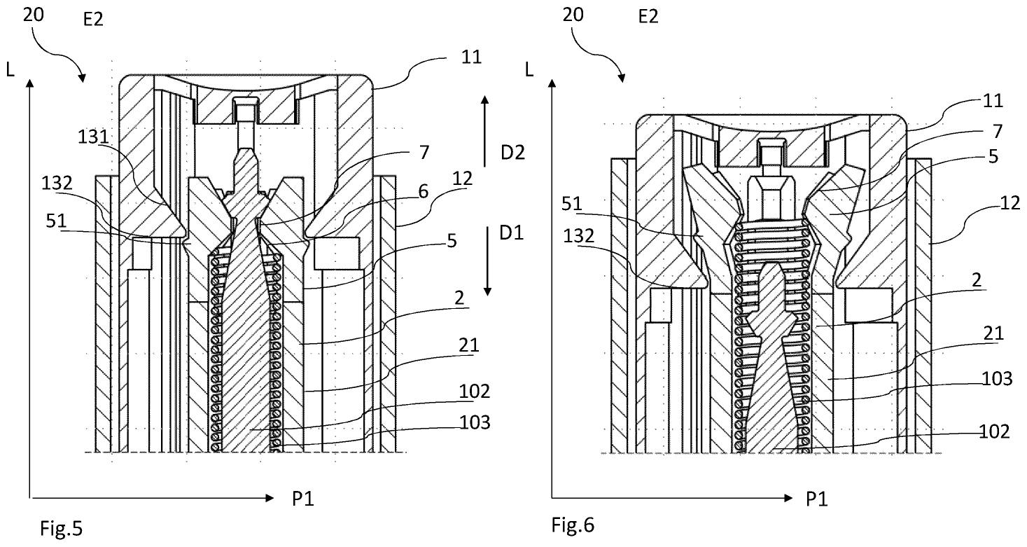

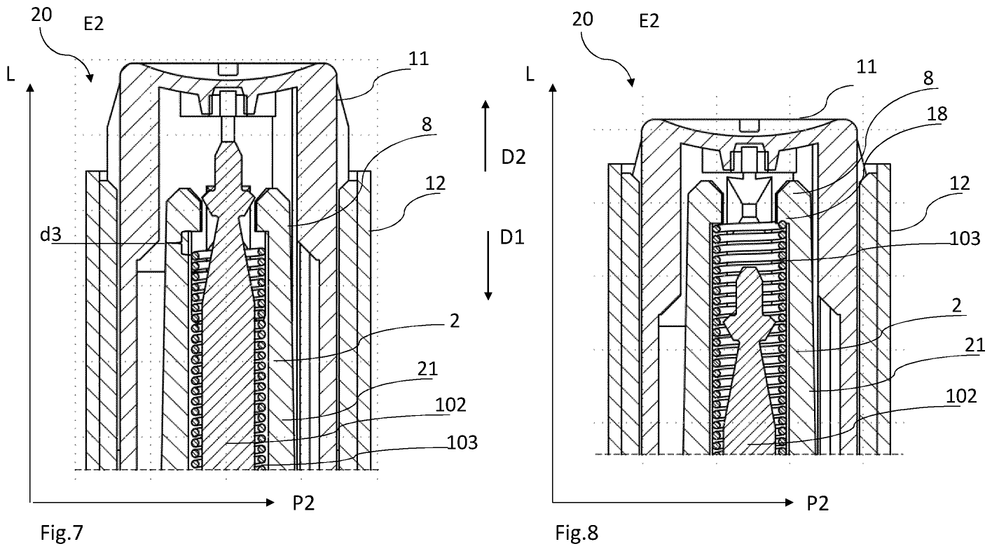

FIG. 2A shows a perspective view of the plane sections P1 and P2 along which the cross-sectional views of the rear assembly 20 are shown in the FIGS. 3 to 8. The planes P1 and P2 are perpendicular to each other and to the longitudinal axis L.

FIG. 2B shows a perspective view of the rear subassembly 20 according to a preferred embodiment of the present disclosure.

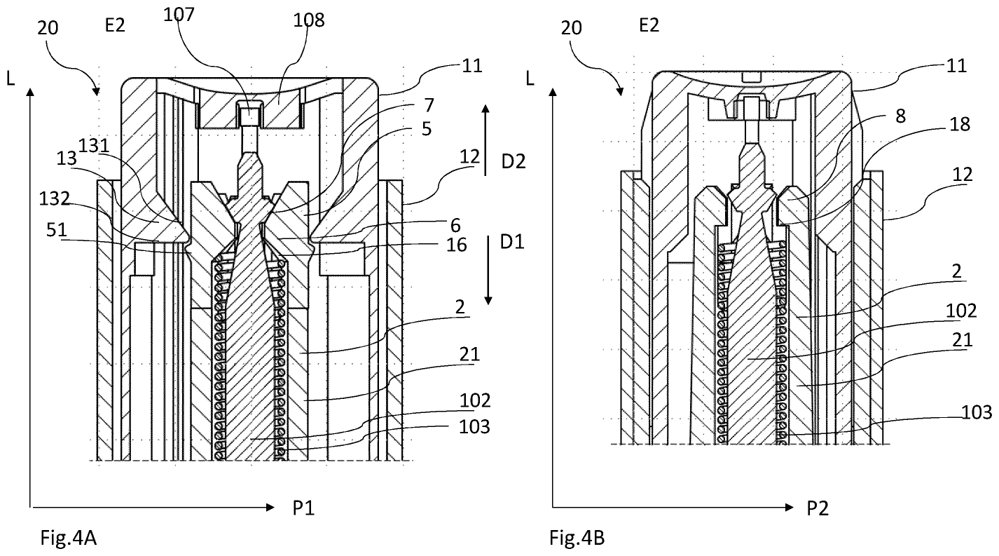

FIG. 4A shows a cross-sectional view of the rear subassembly 20 along the cross-sectional plane P1. As is shown in FIG. 4A, the activation button 11 is at least partially arranged within the tubular outer body 12 and is movable along the longitudinal axis L. Further the rear subassembly comprises an inner tubular body 21.

The inner tubular body 21 comprises a distal part 2 and a proximal part. The proximal part comprises a medicament delivery member cover contact surface configured to contact a medicament delivery member cover of the medicament delivery device (the proximal part is not shown in the figures). The distal part 2 comprises two flexible arms 5 extending distally. The inclined end surface is a plunger rod support 7 at the distal end of the flexible arm 5. The inclined end surface, i.e. the plunger rod support 7, is radially inwardly inclined in the proximal direction. The degree of inclination of the surface of the plunger rod support 7 may vary in function of the intended activation force required for activation. In other words, an increase or decrease of the force required to push the activation button 11 and thus activate the drive spring may vary as a function of the degree of inclination of the plunger rod support 7. As exemplarily shown in FIGS. 3,5 and 6, the plunger rod support 7 radially inwardly protrudes from the flexible arm 5. The two flexible arms 5 further each comprise a first spring support 6, wherein each first spring support 6 comprises a protrusion or step extending radially inwards and having a proximally facing first spring support surface 16 configured to support the distal end of a drive spring of the medicament delivery device, wherein the first spring support surface 16 is radially inwardly inclined in the proximal direction. Likewise, the degree of inclination of the surface of the first spring support surface 16 may vary in function of the intended activation force required for activation. In other words, an increase or decrease of the force required to push the activation button 11 and thus activate the drive spring may vary also as a function of the degree of inclination of the surface of the first spring support surface 16.

In an alternative example, the distal part 2 comprises a single flexible arm 5 extending distally.

The flexible arm comprises an inclined end surface at the distal end of the flexible arm. The inclined end surface, i.e. a plunger rod support 7, is radially inwardly inclined in the proximal direction. The degree of inclination of the surface of the plunger rod support 7 may vary in function of the intended activation force required for activation. In other words, an increase or decrease of the force required to push the activation button 11 and thus activate the drive spring may vary as a function of the degree of inclination of the plunger rod support 7. As exemplarily shown in FIGS. 3, 5 and 6, the plunger rod support 7 radially inwardly protrudes from the flexible arm 5. The flexible arm 5 further comprises a first spring support 6, wherein the first spring support 6 comprises a protrusion or step extending radially inwards and having a proximally facing first spring support surface 16 configured to support the distal end of a drive spring of the medicament delivery device, wherein the first spring support surface 16 is radially inwardly inclined in the proximal direction. Likewise, the degree of inclination of the surface of the first spring support surface 16 may vary in function of the intended activation force required for activation. In other words, an increase or decrease of the force required to push the activation button 11 and thus activate the drive spring may vary also as a function of the degree of inclination of the surface of the first spring support surface 16. The distal part 2 further comprises second spring supports 8 extending distally from the inner tubular body 21. The second spring supports 8 and the flexible arm 5 are facing each other i.e. the arms are opposite to each other.

The activation button 11 comprises further radially inwardly extending protrusions 13 having radially inwardly inclined surfaces 131, the inclined surfaces facing in the distal direction D2. The protrusions 13 each comprise a step extending radially inwards and having a proximally facing support surface 132 configured to support a radially outwardly extending protrusion 51 of the flexible arms 5. Alternatively, a surface of the protrusions 13 abuts an outer surface of the flexible arms 5 such that the contact of the surfaces creates a friction force which can prevent an accidental activation of the activation button 11 without requiring the presence of protrusions 51.

The distal part 2 further comprises two second spring supports 8, each extending distally from the inner tubular body 21. As shown in FIGS. 4A to 8, the second spring supports 8 and the flexible arms 5 are distributed alternatingly around the circumference of the distal part, with a slot i.e. a spacing therebetween. Spacing apart the flexible arms 5 and the spring supports kinematically decouples them from each other, in other words the forces applied on the flexible arm 5 do not affect the second spring support 8, allowing the flexible arm 5 to move relative to the second spring supports 8.

As further shown in FIGS. 4A,4B, 7 and 8, each spring support 8 comprises a protrusion or step extending radially inwards and having a proximally facing second spring support surface 18 configured to support the distal end of a drive spring of the medicament delivery device.

The shown rear subassembly 20 further comprises a plunger rod 102 coaxially arranged within the inner tubular body 21 and releasably connected to the plunger rod support 7 of the flexible arms 5 of the distal part 2. The shown rear subassembly 20 further comprises a drive spring 103 coaxially arranged between the plunger rod 102 and the second spring supports 8 of the inner tubular body 21, which is configured to proximally displace the plunger rod 102 when the plunger rod 102 is released from its engagement with the plunger rod support 7 of the distal part 2 due to a deflection of the flexible arms 5. In FIG. 8, the drive spring 103 is shown in an expanded state and so the plunger rod 102 is displaced in the proximal direction D1.

In an initial, non-activated state S1 as shown in the FIGS. 3A, 3B and 4A, the distal end of the drive spring 103 is supported by the first spring support 6 of the flexible arms 5 and the proximal end of the drive spring 103 is supported by a proximal part of the plunger rod 102, for example a proximal ledge of the plunger rod 102 (not shown in the figures). In an activated i.e. final state S2 as shown in FIGS. 3D, 3E, 6 and 8 when the flexible arms 5 are flexed outwardly, the drive spring is free to move in the distal D2 and proximal D1 directions, and the distal end of the drive spring 103 is supported by the second spring support surfaces 18 of the second spring support 8, and the proximal end of the drive spring 103 is supported by a proximal part of the plunger rod 102, for example a proximal ledge of the plunger rod 102 (not shown in the figures).

The second spring support surface 18 of the second spring support 8 is positioned further apart in the distal direction D2 with respect to the first spring support surface 16 of the first spring support 6 such that when in the non-activated state S1 the distal end of the drive spring 103 is held by the first spring support surface 16 and the distal end of the spring 103 is spaced apart by a distance d3 from the second spring support surface 18. When in an activated state S2 the outwardly flexed flexible arms 5 allow the drive spring 103 to move in the distal direction D2 until the distal end of the drive spring 103 abuts the second spring support surface 18, the distal end of the drive spring 103 has been displaced by the distance d3 in distal direction D2. Thereby the distal end of the drive spring exerts a radially outwardly directed force on the flexible arms 5 such that the flexible arms 5 are further pushed outwardly. In this state the flexible arms 5 abut the inwardly inclined surfaces 131 of the inwardly extending protrusions 13 of the activation button 11 such that the activation button is held in place and is hindered from movement in the distal direction D2. Accordingly, a rattling sound by a movement of the activation button 11 can be avoided.

A play of the activation button 11 in proximal direction D1, and thus an accidental activation, is prevented by the outwardly extending protrusions 51 of the flexible arms 5, which abut the proximally facing support surface 132 of the protrusions 13.

The activation button 11 comprises an optional guide hole 107 and a displacement structure 108. As shown in FIG. 4A, the guide hole 107 is configured to axially guide the distal end of the plunger rod 102 when displaced. In this position, the displacement structure 108 interacts with the inclined end surface 6 of the flexible arms 5 and thereby deflects the flexible arms 5 radially outwardly, such that the distal end of the plunger rod 102 is released from the plunger rod support 7 as shown in FIG. 6.

According to an alternative embodiment, the displacement structure 108 is radially inclined with the same angle of inclination as the inclined end surface 6 of the flexible arms 5.

According to an alternative embodiment, the flexible arms 5 flex radially outwardly when the protrusions 13 are displaced proximally such that the edge of the protrusions 13 is moved beyond the protrusions 51 of the flexible arms 5 such that the flexible arms 5 are free to flex outwardly as shown in FIG. 6. In this example the flexible arms 5 are configured to be in a pretensioned state and to be held in position by the engagement of the protrusions 13 of the activation button 11 and the protrusion 51 of the flexible arms 5.

The medicament delivery device 10 according to a preferred embodiment of the present disclosure is shown in FIG. 1 and comprises a distal rear subassembly 20 and a proximal front assembly 14 (see FIG. 1). The distal rear subassembly 20 is a subassembly 20 according to one of the embodiments described in the foregoing paragraphs.

According to an embodiment, the front assembly 14 comprises a proximal housing 141 connectable to the distal housing 121 of the rear subassembly 20 and a displaceable medicament delivery member cover, for example a needle cover, partially arranged within the proximal housing 141.

According to an embodiment, the front assembly 14 further comprises a rotator (not shown in the figures) coaxially connected to the distal inner part of the proximal housing 141. The rotator is movable in relation to the proximal housing 141. According to an embodiment, the front assembly 14 further comprises a medicament container holder (not visible in the figures) connected via the medicament delivery member cover and the rotator to the proximal housing 141.

According to an embodiment, the front assembly 14 further comprises an optional medicament delivery member cover spring disposed between the medicament delivery member cover and the medicament container holder and configured to apply a biasing force on the medicament delivery member cover. The medicament delivery member cover is axially movable inside at least the proximal housing 141.

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary medicaments that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a medicament as listed herein (or a pharmaceutically acceptable salt of the medicament) may include one or more other active ingredients, or may be the only active ingredient present.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

1-15. (canceled)

16. An inner tubular body for a medicament delivery device, wherein the inner tubular body extends along a longitudinal axis from a proximal end to a distal end, the inner tubular body comprising:
a distal part and a proximal part,
wherein the distal part comprises a flexible arm extending distally, wherein the flexible arm comprises a first spring support at the distal end of the flexible arm and a plunger rod support at the distal end of the flexible arm, wherein the first spring support is inclined in a radially inward direction and is configured to support a distal end of a drive spring of the medicament delivery device when the medicament delivery device is in a non-activated state, the distal part further comprising a second spring support extending distally and being configured to support the distal end of the drive spring of the medicament delivery device when the medicament delivery device is in an activated state,
wherein the plunger rod support is configured to engage with a plunger rod of the medicament delivery device in order to inhibit a proximal movement of the plunger rod when the medicament delivery device is in the non-activated state, and
wherein the first spring support is configured to interact with an activation button that is at a distal end of the medicament delivery device, with triggering of said activation button allowing a radial outward deflection of the flexible arm thereby disengaging the plunger rod support from the plunger rod and disengaging the first spring support from the distal end of the drive spring.

17. The inner tubular body of claim 16, wherein the flexible arm, and the second spring supports are spaced apart so that the flexible arm can move relative to the second spring supports.

18. The inner tubular body of claim 16, wherein the second spring support comprises a second spring support surface, spaced apart in the distal direction with respect to a first spring support surface of the first spring support such that, when in the non-activated state, the distal end of the drive spring is supported by the first spring support surface and the distal end of the drive spring is spaced apart by a distance from the second spring support surface.

19. The inner tubular body of claim 16, wherein when in the activated state the flexible arm is configured to flex outwardly and disengage the drive spring in the distal direction such that the distal end of the drive spring abuts the second spring support surface thereby the distal end of the drive spring has been displaced by the distance in the distal direction.

20. The inner tubular body of claim 16, wherein each plunger rod support has a radially inwards extending protrusion configured to form a stop surface for engagement with a distal portion of the plunger rod.

21. The inner tubular body of claim 16, wherein each first spring support comprises a protrusion or step extending radially inwards and having a proximally facing support surface configured to support the distal end of the drive spring.

22. The inner tubular body of claim 16, wherein when in the activated state the distal end of the drive spring exerts a radially outwardly directed force on the flexible arm such that the flexible arm is further pushed outwardly, thereby abutting inwardly inclined surfaces of inwardly extending protrusions of an activation button such that the activation button is prevented from moving in the distal direction.

23. The inner tubular body of claim 16, wherein the flexible arm and the at least two second spring supports are alternately arranged in the circumferential direction.

24. A rear subassembly for a medicament delivery device, the rear subassembly comprising:
a distal housing;
the inner tubular body according to claim 16, wherein the tubular body is coaxially arranged within the distal housing;
a plunger rod coaxially arranged within the inner tubular body and releasably connected to the plunger rod support of the inner tubular body;
a drive spring coaxially arranged between the plunger rod and second spring supports of the inner tubular body, wherein a distal end of the drive spring is supported by the first spring support when in a first non-activated state or the distal end of the drive spring is supported by the second spring supports when in a second activated state and a proximal end of the drive spring is supported by a proximal ledge of the plunger rod;
an activation button connected to the distal housing, wherein the activation button comprises a radially inwardly extending protrusion, the radially inwardly extending protrusion comprising a radially inwardly inclined surface, wherein the inclined surface is facing in the distal direction, the protrusion comprising a step extending radially inwards, the step having a proximally facing support surface configured to support a radially outwardly extending protrusion of the flexible arm when the medicament delivery device is in the non-activated state; and
a distal spring arranged between the activation button and the inner tubular body, wherein the distal spring is configured to apply a biasing force on the inner tubular body.

25. The rear subassembly of claim 14, wherein the activation button comprises a guide hole along the longitudinal axis of the medicament delivery device configured to axially guide the distal end of the plunger rod when the inner tubular body is distally displaced.

26. The rear subassembly of claim 14, wherein the activation button comprises a displacement structure configured to interact with an inclined distal end surface of the flexible arm in order to radially deflect the flexible arm such that the plunger rod support is disengaged from the plunger rod when the inner tubular body is distally displaced.

27. The rear subassembly of claim 26, wherein the displacement structure is rigid and is radially inclined, preferably with the same angle of inclination as the inclined end surface of the flexible arm.

28. A medicament delivery device comprising a distal subassembly and a proximal subassembly, wherein the distal subassembly is a rear subassembly according to claim 24.

29. The medicament delivery device of claim 28, wherein the proximal subassembly comprises:
a proximal housing connectable to the distal housing;
a rotator coaxially connected to a distal inner part of the proximal housing and movable in relation to the proximal housing;
a displaceable medicament delivery member cover partially arranged within the proximal housing;
a medicament container holder connected to the medicament delivery member cover and configured to receive a medicament container; and
a medicament delivery member cover spring disposed between the medicament delivery member cover and the medicament container holder and configured to apply a biasing force to the medicament delivery member cover,
wherein the medicament delivery member cover is axially movable inside at least the proximal housing such that a distal end of the medicament delivery member cover contacts the contact surface of the inner tubular body whereby the inner tubular body is distally displaced.

30. The medicament delivery device of claim 28, wherein the medicament delivery device is a disposable single-dose auto-injector.
